(19) United States
(12) Patent Application Publication
(10) Pub. No.: US 2026/0242727 A1
(43) Pub. Date: Aug. 20, 2026

(54) METHOD FOR PRODUCING THREE-DIMENSIONALLY CULTERED SKIN MODEL COMPRISING HAIR FOLLICLE STRUCTURE USING FIBROBLASTS AND THREE-DIMENSIONALLY CULTERED SKIN MODEL PRODUCED THEREBY

(71) Applicant: Tego Science Inc., Seoul (KR)

(72) Inventors: Saewha JEON, Seoul (KR); Huynah MOON, Seoul (KR); Kipunsam LEE, Seoul (KR)

(73) Assignee: Tego Science Inc., Seoul (KR)

(21) Appl. No.: 19/564,745

(22) Filed: Mar. 12, 2026

Related U.S. Application Data
(63) Continuation-in-part of application No. PCT/KR2024/013287, filed on Sep. 4, 2024.

(30) Foreign Application Priority Data
Sep. 14, 2023 (KR) 10-2023-0122435

(51) Int. Cl. C12N 5/071

(52) U.S. Cl. CPC C12N 5/0629; C12N 2501/998; C12N 2501/999; C12N 2503/06; C12N 2506/094; C12N 2506/1307; C12N 2513/00

(57) ABSTRACT

A method for producing a three-dimensionally cultured skin model of a hair follicle structure using fibroblasts, and a three-dimensionally cultured skin model are described. Dermal papilla cells are replaced with unique fibroblasts that exhibit gene expression similar to the gene expression exhibited by dermal papilla cells when the dermal papilla cells are treated with a hair growth promoting material to produce a hair follicle structure.

### CROSS-REFERENCE TO RELATED APPLICATIONS

This application is a bypass continuation-in-part of International Application No. PCT/KR2024/013287, filed Sep. 4, 2024, which was published in the Korean language on Mar. 20, 2025, under International Publication No. WO 2025/058312A1, which claims priority under 35 U.S.C. § 119(b) to Korean Application No. 10-2023-0122435, filed Sep. 14, 2023, the disclosures of which are incorporated herein by reference in their entirety.

### TECHNICAL FIELD

The present invention relates to a method for producing a three-dimensionally cultured skin model including a hair follicle structure using fibroblasts, and a three-dimensionally cultured skin model produced thereby.

### BACKGROUND ART

The skin is the largest organ covering the entire surface of the human body and has various functions, including regulating the body temperature and acting as a barrier against the external environment, and in the case of adults, its area ranges from about 1.2 m² to 2.3 m². The skin is composed of the epidermis and the dermis, the dermal layer is composed of collagen and fibroblasts, and the epidermal layer is composed of keratin cells, melanocytes, and the like. Compared to the epidermal layer having a relatively uniform thickness, the thickness of the dermal layer is known to vary greatly depending on the region, but is known to have a thickness about threefold that of the epidermis.

The epidermal layer is stratified into the basal membrane, spinous layer, granular layer, and stratum corneum, and multiple layers of terminally differentiated dead cells are piled up on the skin surface to perform a protective membrane function. The basal membrane is in the form of a thin membrane, in which laminin and several extracellular matrices are deposited on a type 4 collagen lattice, and the cells of the basal layer are attached thereto. When a wound occurs, the wound is accompanied by decomposition of the basal membrane, the cells of the basal layer come into contact with type 1 collagen of the dermis, and in this case, epidermal cells migrate laterally to induce re-epithelialization.

The dermal layer is composed of the papillary dermis, in which fibroblasts are abundant and microvessels are distributed, and the reticular dermis in which collagen fibers are abundant. The hair follicles and several skin appendages are located deep in the dermis, causing epidermal cells to grow from the hair follicles to induce re-epithelialization even when the epidermal layer is lost, such as in the case of partial thickness wounds.

Hair follicles primarily serve a cosmetic function in social life rather than a life-sustaining function. Normal hair follicles grow and are maintained through repeated hair cycles of the anagen (3 to 5 years) in which the division of hair matrix cells is activated and hair grows, the catagen (3 weeks) in which hair growth stops, the dermal papilla begins to be separated from the hair in the hair follicle, and the hair follicle structure begins to degenerate, and the telogen (3 months) in which the dermal papilla and hair separate from each other and the hair falls out.

However, in modern life, alopecia is caused by factors such as abnormal hormone secretion, excessive stress, and environmental pollution, causing significant psychological distress in people's social lives. In this regard, in the past, passive methods such as ignoring alopecia or covering the symptoms through wigs were used, but in recent years, as research into the biology and molecular biology of hair follicles and alopecia has progressed, new facts have been revealed one after another, and hair follicles are emerging as a new core field of research while the presence of stem cells has also become known.

To perform appropriate treatments and test efficacies of active ingredients according to the overall progression and state of alopecia, a deeper understanding of the complex structure of hair follicles and their regulatory mechanisms is required, and an in vitro system that allows reproducible and quantitative observation and artificial adjustment of various factors is required. To date, murine vibrissae have been mainly used in research on hair follicles, and there have been very few studies using human hair follicles. Unlike rodents, which shed their hair at a predetermined cycle, human hair follicles each have an independent cycle, leading to different tendencies for growth factors and many biological characteristics and functional differences. Therefore, in order to accurately understand and study the characteristics of human hair follicles, it is desirable to perform research using human hair follicle cells.

In particular, although research is being conducted on the creation of artificial skin using dermal papilla cells, dermal papilla cells, which are involved in hair production and hair follicle formation, have a problem in that it is difficult to culture cells because an isolation method different from general cells is applied, the inherent characteristics of the dermal papilla cells are easily lost during in vitro culture, and the ability of the dermal papilla cells to proliferate deteriorates.

Further, there are limitations in the tests that are conducted to evaluate the efficacy and safety of alopecia treatment drugs, cosmetics, and pharmaceuticals using dermal papilla cells.

Therefore, there is an urgent need for producing a three-dimensionally cultured skin model capable of replacing dermal papilla cells, which are difficult to isolate and culture.

### RELATED ART DOCUMENTS

### Patent Documents

Korean Patent Application No. 10-2016-0007901

### Non-Patent Documents

Hasan Erbil Abaci et al. Tissue engineering of human hair follicles using a biomimetic developmental approach, NATURE COMMUNICATIONS |(2018) 9:5301|

### DISCLOSURE

### Technical Problem

Therefore, as a result of research efforts to solve the above-described problems, the present inventors discovered unique fibroblasts that exhibit gene expression similar to that of dermal papilla cells when dermal papilla cells are treated with a hair growth promoting substance, for reproducing a hair follicle structure similar to that of a three-dimensionally cultured skin including a hair follicle structure using dermal papilla cells, and thereby completed the present invention by developing a three-dimensionally cultured skin model including a hair follicle structure using fibroblasts that can be easily mass-cultured in place of dermal papilla cells which are difficult to isolate and culture.

### Technical Solution

Therefore, an object of the present invention is to provide a method for producing a three-dimensionally cultured skin model including a hair follicle structure using fibroblasts that exhibit gene expression similar to that of dermal papilla cells treated with a hair growth-promoting substance, wherein the fibroblasts can be easily mass-cultured in place of dermal papilla cells which are difficult to isolate and culture.

Furthermore, another object of the present invention is to provide a three-dimensionally cultured skin model including a hair follicle structure using fibroblasts that exhibit gene expression similar to that of dermal papilla cells treated with a hair growth-promoting substance.

### Advantageous Effects

In the present invention, a specific fibroblast (HF1) that exhibits gene expression similar to that of dermal papilla cells obtained by treating dermal papilla cells isolated from human scalp tissue with a hair growth-promoting substance was selected, and when fibroblasts were used to produce a three-dimensionally cultured skin including a hair follicle structure, similar to that of a three-dimensionally cultured skin including a hair follicle structure using dermal papilla cells was reproduced.

Accordingly, by replacing dermal papilla cells, which are difficult to isolate and culture with fibroblasts that can be easily mass-cultured, a three-dimensionally cultured skin model including a hair follicle structure using fibroblasts can be produced.

### DESCRIPTION OF DRAWINGS

FIG. 1 illustrates the results of gene expression of two types of fibroblasts, HF1 and HF2, one type of dermal papilla cell, and dermal papilla cells treated with a hair growth promoting substance through heatmap analysis.

FIG. 2 illustrates the expression of eight genes that exhibit high expression in HF1 among dermal papilla cells treated with a hair growth promoting substance and two types of fibroblasts, HF1 and HF2, through heatmap analysis.

FIG. 3 illustrates the results of treating HF1, HF2 and DP cells with minoxidil for 24 hours and then performing polymerase chain reaction (real time RT-PCR).

FIG. 4 illustrates the western blot results of analyzing the expression of ITGA2, TBX3, and ITGA6 genes in HF1, HF2, and DP cells after treating the HF1, HF2, and DP cells with minoxidil for 24 hours.

FIG. 5 schematically illustrates a dermal structure with cylindrical holes.

FIG. 6 illustrates a three-dimensionally cultured skin having a dermal layer and an epidermal layer including a hair follicle structure, produced using fibroblasts, as shown by hematoxylin and eosin (H&E) tissue staining.

FIG. 7 shows a three-dimensionally cultured skin having a dermal layer and an epidermal layer including a hair follicle structure, produced using HF1 cells, HF2 cells, and DP cells, as confirmed by hematoxylin and eosin (H&E) tissue staining.

FIG. 8 shows, by hematoxylin and eosin (H&E) tissue staining, that a three-dimensionally cultured skin model including the dermal layer, an epidermal layer, and hair follicle structure was reproduced using HF1 that exhibits high expression of eight genes similar to that of dermal papilla cells treated with a hair growth promoting substance.

### BEST MODE

The present invention relates to a method for producing a three-dimensionally cultured skin model including a hair follicle structure using fibroblasts that exhibit gene expression similar to that of dermal papilla cells treated with a hair growth promoting substance.

Specifically, the method for producing a three-dimensionally cultured skin model including a hair follicle structure according to the present invention includes:
1) selecting fibroblasts that exhibit gene expression similar to that of dermal papilla cells treated with a hair growth-promoting substance;
2) inoculating a cell mixture comprising fibroblasts and collagen into a cell culture device, fixing a cylindrical hole-forming tool, and allowing the cell mixture to solidify to produce a dermal construct;
3) removing the cylindrical hole-forming tool from the structure formed in step 2) to form a cylindrical hole;
4) inoculating the fibroblasts or dermal papilla cells selected in step 1) into the cylindrical hole formed in step 3) to produce a dermal layer;
5) inoculating keratinocytes onto the upper layer of the dermal layer to culture the keratinocytes until a cell sheet is formed; and
6) exposing the cell sheet to air and culturing the cell sheet to form an epidermal layer.

Hereinafter, the production method of the present invention will be described in detail as follows.

The term “hair follicle” as used herein refers to a skin appendage found only in mammals, and the hair follicle is formed from the embryonic stage by epithelial-mesenchymal interactions. During the embryonic stage, hair follicle development is initiated by a first dermal signal, and results in the formation of a placode as the epithelial layer thickens. The epithelial signal released from this thickened epithelial plate induces the dermal condensation of mesoderm-derived dermal cells, and the formed condensate again releases a second dermal message. This signal promotes the proliferation of epithelial cells and simultaneously induces the invasion of the epidermal cells into the dermis to surround the condensates, resulting in the formation of a dermal papilla. The initial hair follicle structure is generated in this manner, and thus develops into a mature hair follicle that forms hair while the proliferation and differentiation of epithelial cells continue. In mature hair follicles, interaction between hair follicle matrix cells and dermal papilla cells leads to specialized differentiation of hair matrix cells, resulting in the production and growth of hair.

The term “hair growth promoting substance” as used herein refers to a substance that prevents alopecia or promotes hair growth, and specific examples thereof include minoxidil and finasteride, which are FDA-approved drugs that promote hair growth, CHIR99021 and CHIR98014, which are Wnt signaling activating compounds generally involved in hair growth, and the like.

The term “dermal papilla cells” as used herein refers to the core cells responsible for hair growth, which are located at the bottom of the hair root and are surrounded by hair matrix cells as in forming a castle wall. Dermal papilla cells are cells that produce hair in the tissue, and are present beneath the hair follicle, absorb necessary nutrients from the capillaries that flow to the dermal papilla cells, produce hair while dividing and proliferating, and determine the number, thickness, and regrowth cycle of the hair.

The term “fibroblasts” as used herein refers to cells that constitute the important components of fibrous connective tissue, and particularly to the main cells that constitute the dermal layer in the skin that is relevant to the present invention. In the present invention, fibroblasts may be derived from humans, mice, rats, and the like, but are not limited thereto. For the purposes of the present invention, the fibroblasts used in producing the dermal layer in the process of producing the three-dimensionally cultured skin model of the present invention may be, in particular, derived from humans.

The term “collagen” as used herein refers to a scleroprotein present in both plants and animals, and is mainly present in the bones and skin of animals, is also distributed in cartilage, organ membranes, hair, and the like, and is also called collagenous material. Collagen is a protein with a triple helix structure, and has non-helical telopeptides at both ends of the triple helix structure. In the present invention, collagen may be derived from humans, cows, pigs, mice, rats, and the like, but is not limited thereto. In the present invention, collagen may be not only natural collagen but also collagen modified by biotechnology or modified by biotechnological or chemical treatment, but there is no limitation on its origin or whether it has been modified or not.

The term “keratinocytes” as used herein refers to cells that have the ability to keratinize among epidermal cells and make up the majority of epidermal cells, and generally refers to cells which are the same as keratin cells. In the present invention, keratin cells may be derived from humans, mice, rats, and the like, but are not limited thereto. For the purpose of the present invention, the keratin cells used to produce the epidermal layer in the process of producing the three-dimensionally cultured skin model of the present invention may be, in particular, human-derived keratin cells, and may be keratin cells derived from normal tissue or diseased tissue such as chemicals, burns, wounds, scars, and ulcers, but are not limited thereto.

The term “three-dimensionally cultured skin model” as used herein refers to a skin layer model that similar to a natural skin layer and is produced by an artificial culture process that is not collected or obtained from an animal, and the like. The origin of the cells which constitute it is not limited.

According to an embodiment of the present invention,

First, to reproduce artificial skin with a hair follicle structure, fibroblasts that exhibit gene expression similar to that of dermal papilla cells treated with a hair growth promoting substance are selected.

As a result of heatmap analysis, it was found that genes whose expression is similar to those of dermal papilla cells treated with the hair growth-promoting substance exhibit similar expression patterns between dermal papilla cells and fibroblasts when the dermal papilla cells are treated with the hair growth-promoting substance. The gene whose expression is similar to those of dermal papilla cells treated with the hair growth-promoting substance may be one or more selected from the group consisting of AFF1, KCTD12, KANK1, NFAT5, RAPGEF6, ITGA2, TBX3, and ITGA6, and preferably one or more selected from the group consisting of ITGA2, TBX3, and ITGA6.

In one embodiment, fibroblasts may be selected based on expression levels of eight genes selected from the group consisting of AFF1, KCTD12, KANK1, NFAT5, RAPGEF6, ITGA2, TBX3, and ITGA6.

For example, fibroblasts may be selected when the expression of all eight genes is increased by at least 1.25-fold relative to dermal papilla cells treated with minoxidil (DP+minoxidil).

In one embodiment, according to the experimental data shown in FIG. 3, the increase of HF1 in gene expression relative to DP+minoxidil ranged from about 1.61±0.17-fold to about 15.98±1.78-fold.

In step 2), a cell mixture comprising fibroblasts and collagen is inoculated into a cell culture device, and the cell mixture is allowed to solidify while fixing a cylindrical hole-forming tool to produce a dermal construct.

The cell culture device may be a container capable of cell culture, specifically, a transwell insert or the like, but is not limited thereto.

The cylindrical hole-forming tool may be a needle, a syringe, a pipette tip, an aluminum mold, a stainless steel mold, and the like, which can be guided to produce a circular hole in the dermal structure in order to mimic the hair follicle structure.

In this case, the dermal structure including fibroblasts and collagen may include mouse-derived or human-derived fibroblasts at 1×10⁴ to 1×10⁷ cells per mL, and may additionally include one or more cells selected from the group consisting of endothelial cells, adipocytes, and mesenchymal stem cells.

In step 3), a plurality of cylindrical holes are formed in the dermal structure formed above.

The cylindrical holes are intended to mimic the structure of hair follicles in the skin, it is desirable to fabricate the cylindrical holes in a shape as similar to the size of a hair follicle as possible, and specifically, the circular hole has a diameter of 100 μm to 1000 μm, and the cylindrical length is 200 μm to 500 μm. The spacing between holes may be 100 μm to 1000 μm, but is not limited thereto.

The inoculating of a composition including fibroblasts and collagen into a culture dish and hardening the composition corresponding to step 2) above is performed for 10 hours to 48 hours (10 hours to 24 hours).

In step 4), the fibroblasts or dermal papilla cells selected in step 1) above are inoculated into the cylindrical holes to produce a dermal layer including a hair follicle structure.

In this case, the selected fibroblasts or dermal papilla cells may be inoculated at 1×10 t 1×10⁶ cells per cylindrical hole.

Step 4) above may be performed for 1 hour to 48 hours.

In step 5), keratinocytes are inoculated onto the upper layer of the dermal layer to culture the keratinocytes until a cell sheet is formed.

In this case, it is desirable to inoculate the keratinocytes prepared above at 1×10⁴ to 1×10⁷cells per cm² of dermal layer.

Further, the keratinocytes may be keratin cells derived from skin, mucous membranes, cornea, hair follicles or epithelial tissue, but are not limited thereto. The keratin cells may additionally include one or more cells selected from the group consisting of melanocytes, Merkel cells, and Langerhans cells during inoculation.

Furthermore, step 5) above may be performed for about 2 days to 21 days, but this may vary depending on the keratin cells to be inoculated, the area of the dermal layer, and the like, and thus is not particularly limited.

In step 6), the cell sheet is exposed to air and cultured to form an epidermal layer, thereby producing a three-dimensionally cultured skin model.

Step 6) above may be performed for 7 days to 21 days.

The present invention also includes a three-dimensionally cultured skin model produced by the above method.

The present invention also includes a three-dimensionally cultured skin model comprising a dermal layer prepared from a composition comprising collagen and fibroblasts that exhibit gene expression similar to that of dermal papilla cells treated with a hair growth-promoting substance, and an epidermal layer formed by keratinocytes.

### MODES OF THE INVENTION

Hereinafter, the present invention will be described in more detail with reference to the following Examples. However, the following Examples are only for exemplifying the present invention, and the scope of the present invention is not limited thereto.

### Example 1: Selection of Fibroblast HF1 Cells That Exhibit Gene Expression Similar to Dermal Papilla Cells

Dermal papilla cells were isolated by microdissection from human scalp tissue obtained from donors and inoculated into a culture dish, and dermal papilla cells (DP) were isolated from DMEM culture medium containing 10% FBS and cultured.

Fibroblasts were isolated from skin tissue obtained from donors and cultured in F12 culture medium containing 10% FBS.

Dermal papilla cells were inoculated at 2.2×10⁵ cells in a 60-mm diameter culture dish and cultured for 24 hours, the cells were treated with 50 μM minoxidil, 100 μM finasteride, and 5 μM CHIR99021, which are hair growth promoting substances, for 24 hours each and cultured, and fibroblasts were inoculated at 1.2×10⁵ cells in a 60-mm diameter culture dish and cultured for 48 hours.

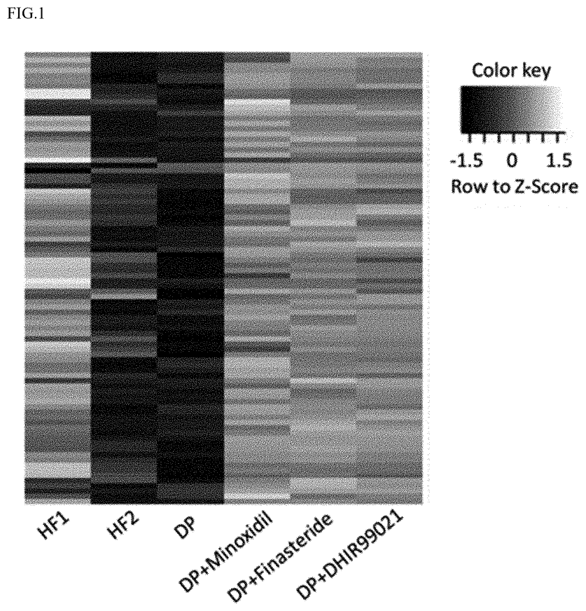

After culture was completed, RNA was extracted from each harvested cell using TRIzol and subjected to mRNA sequencing analysis. Gene expression was analyzed through inter-group mRNA sequencing results for two types of fibroblasts (HF1, HF2), one type of dermal papilla cell (DP), and one type of dermal papilla cell treated with a hair growth promoting substance, it was confirmed that gene expression in one HF of the two types of fibroblasts was similar to that in dermal papilla cells treated with a hair growth promoting substance, this fibroblast was named HF1, the remaining type of fibroblast was named HF2, and the dermal papilla cells were named DP, and the gene expression results were shown by a heatmap analysis (FIG. 1).

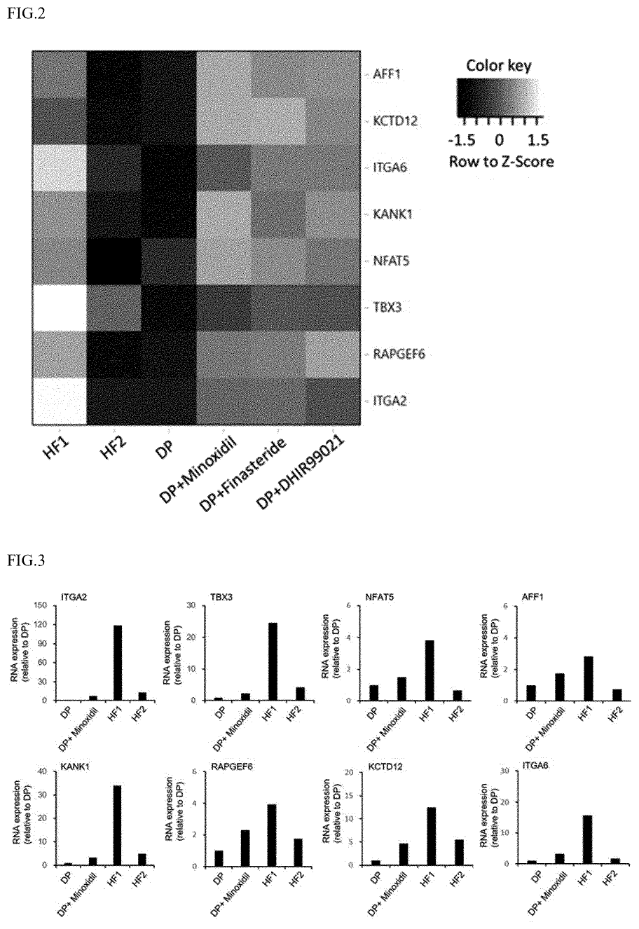

Eight genes (AFF1, KCTD12, KANK1, NFAT5, RAPGEF6, ITGA2, TBX3, and ITGA6) that commonly exhibited a two-fold or higher increase in expression in dermal papilla cells treated with a hair growth promoting substance and in HF1 were selected, and the results of intercellular gene expression for the eight genes were shown by a heatmap analysis (FIG. 2).

When dermal papilla cells were treated with a hair growth promoting substance, it was determined that the genes whose expression levels increased were likely to be genes associated with hair growth.

To analyze the relationship between the eight selected genes and hair growth promotion at the RNA level, RNA was extracted using TRIzol from DP cells, DP cells treated with minoxidil for 24 hours, HF1, and HF 2 cells, and then polymerase chain reaction (real-time RT-PCR) was performed using synthesized cDNA. Polymerase chain reaction was performed using a QuantStudio5 real-time PCR system device by mixing TB Green Premix Ex Taq, ROX dye, and primers designed for the eight genes. As a result, it was confirmed that the expression of all the eight genes was increased in DP cells treated with hair growth promoting substances compared to DP cells, and that the expression was higher in HF1 cells than in HF2 cells. Accordingly, it was confirmed that the expression of the eight genes was increased in DP cells by a hair growth promoting substance, and that the expression of the eight genes was specifically high in HF1 cells at the RNA level (FIG. 3). The expression level of ITGA2 in HF1 was 15.98±1.78-fold higher than DP+minoxidil, whereas that in HF2 was 1.68±0.21-fold. The expression level of TBX3 in HF1 was 10.74±0.16-fold higher than DP+minoxidil, whereas that in HF2 was 1.84±0.18-fold. The expression level of NFAT5 in HF1 was 2.52±0.07-fold higher than DP+minoxidil, whereas that in HF2 was 0.44±0.02-fold. The expression level of AFF1 in HF1 was 1.61±0.17-fold higher than DP+minoxidil, whereas that in HF2 was 0.43±0.01-fold. The expression level of KANK1 in HF1 was 10.29±0.10-fold higher than DP+minoxidil, whereas that in HF2 was 1.51±0.25-fold. The expression level of RAPGEF6 in HF1 was 1.70±0.22-fold higher than DP+minoxidil, whereas that in HF2 was 0.80±0.04-fold. The expression level of KCTD12 in HF1 was 4.93±0.64-fold higher than DP+minoxidil, whereas that in HF2 was 1.17±0.08-fold. The expression level of ITGA6 in HF1 was 4.93±0.44-fold higher than DP+minoxidil, whereas that in HF2 was 0.53±0.01-fold.

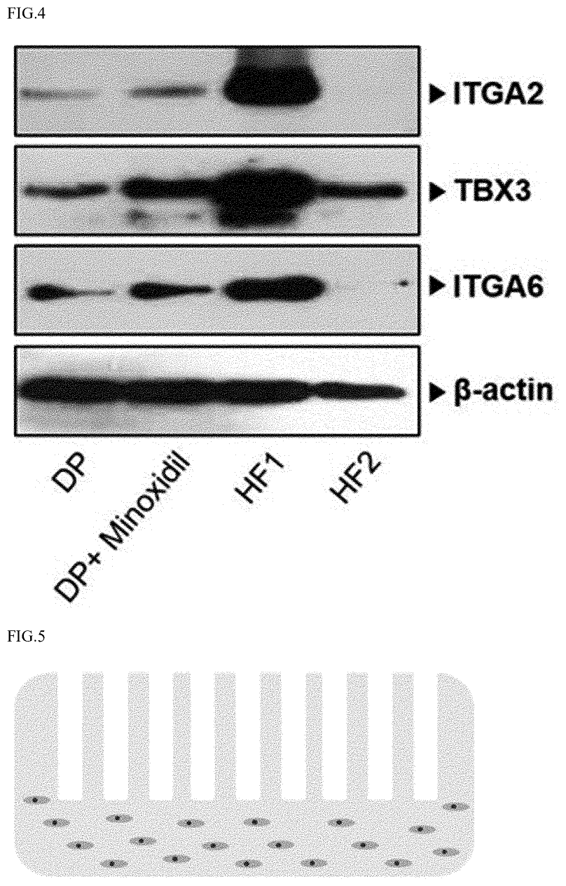

To analyze the relationship between the eight selected genes and hair growth promotion at the protein level, cell lysates were extracted using RIPA buffer from DP cells, DP cells treated with minoxidil for 24 hours, HF1, and HF2 cells, and western blot was performed to observe changes in the expression of ITGA2, TBX3, and ITGA6 genes. As a result, it was confirmed that the expression of all the three genes was increased in DP cells treated with hair growth promoting substances compared to DP cells, and that the expression was higher in HF1 cells than in HF2 cells. Accordingly, it was confirmed that the expression of the three genes was increased in DP cells by a hair growth promoting substance, and that the expression of the three genes was specifically high in HF1 cells (FIG. 4).

Therefore, fibroblast HF1 cells, which have a similar gene expression pattern to dermal papilla cells treated with a hair growth-promoting substance, were used to produce a three-dimensionally cultured skin including a hair follicle structure.

### Example 2: Production of a Three-Dimensionally Cultured Skin Model Including Hair Follicle Structure

### 1) Production of Dermal Layer

After a cell mixed solution was prepared by mixing mouse-derived or human-derived fibroblasts with a 2.0 mg/mL collagen solution and 10× reconstruction buffer (0.2 M sodium bicarbonate, 0.2 M HEPES, and 0.05 N NaOH) such that the cells were included at 1×10⁵ cells per mL, each of 4 mL of the cell mixed solution was added to a 6-well transwell insert, 500 μm diameter needles were fixed at 500 μm intervals in the transwell insert, and the cell mixed solution was hardened in a 10% CO₂ incubator at 37° C. for 24 hours to produce a dermal construct. After 2 hours, the needle was removed from the dermal construct to form a cylindrical hole with a diameter of 500 μm and a height of 3 mm in the dermal construct (FIG. 5).

DP cells and HF1 and HF2 cells were inoculated in an amount of 100 μL per cylindrical structure at 3000 cells into the dermal layer produced above, thereby forming a dermal layer including a hair follicle structure.

### 2) Production of Dermal Layer

2.5×10⁵ human skin-derived keratinocytes were inoculated onto the dermal layer produced in 1) above and cultured. While replacing the medium with a culture medium containing 10 ng/mL EGF every two days, the cells were cultured in a 10% CO₂ incubator at 37° C. for 6 days until a cell sheet was formed.

After the keratinocytes formed a sheet on the dermal layer, the cells were transferred to a 6-well three-dimensional culture container designed for air exposure, the container was filled with a culture medium containing 10 ng/ml of EGF, and then the dermal-epidermal layer produced above was exposed to air. Thereafter, the cells were cultured in a 10% CO₂ incubator at 37° C. for a total of 14 days while replacing the culture medium with a culture medium containing 10 ng/ml EGF every four days.

### 3) Tissue Staining (H&E Staining)

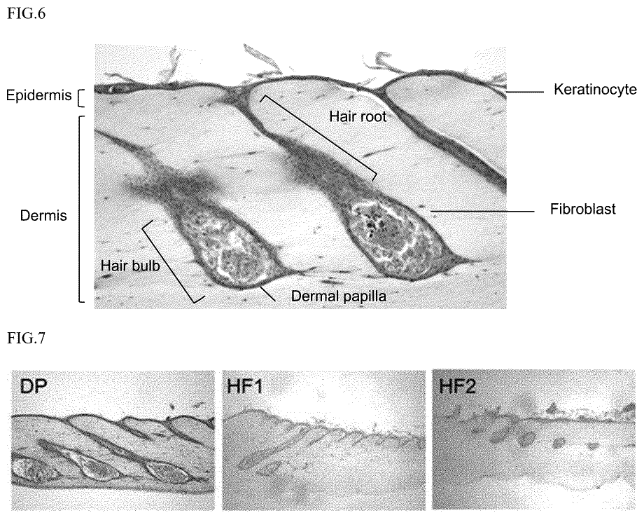

The dermal layer was produced using HF1 cells as described in 1) above, and the epidermal layer was produced as described in 2) above to confirm the structure of a three-dimensionally cultured skin model including a cultured hair follicle structure using hematoxylin and eosin (H&E) tissue staining (FIG. 6). When the skin model was produced using HF1 cells, it was observed that a hair follicle structure including the dermal papilla, hair bulb, and hair root was reproduced.

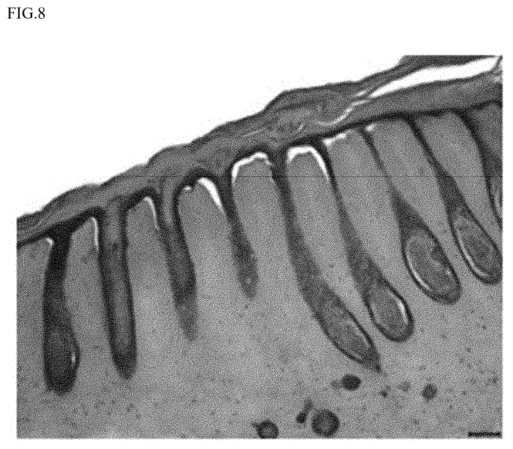

The dermal layer was produced using DP cells and HF1 and HF2 cells as described in 1) above and the epidermal layer was produced as described in 2) above to confirm each of the structures of three-dimensionally cultured skin models including a cultured hair follicle structure by H&E staining, and as a result, a hair follicle structure was observed only in the cultured skin using DP cells and HF1 cells (FIG. 7).

H&E tissue staining was performed through laboratory procedures well known in the art.

When the three-dimensionally cultured skin model was produced using fibroblasts (HF1) that exhibit gene expression similar to dermal papilla cells, it was possible to reproduce a three-dimensionally cultured skin including dermal papilla, hair bulb, and hair root structures, but when the three-dimensionally cultured skin model was produced using fibroblasts (HF2) that do not exhibit specific gene expression, it was not possible to reproduce three-dimensionally cultured skin (FIG. 7).

As a result of confirming the structure of the three-dimensionally cultured skin model (formation of dermal layer+epidermal layer) containing the hair follicle structure produced in Example 2) above by H&E tissue staining, it was confirmed that it was possible to form a basal layer, granular layer, spinous layer, and stratum corneum similar to those of human epidermis and to produce a three-dimensional skin model including a hair follicle structure (FIG. 8).

The present invention is a technology developed through the “Development of 3D cultured skin including hair follicles and animal alternative testing methods” of the 2021 Biomedical Technology Commercialization Support Project (BT210069) sponsored by the Seoul Business Agency of the Seoul Metropolitan Government.

1. A method for producing a three-dimensionally cultured skin model, the method comprising:
1) selecting fibroblasts that exhibit gene expression similar to that of dermal papilla cells treated with a hair growth-promoting substance;
2) inoculating a cell mixture comprising fibroblasts and collagen into a cell culture device, fixing a cylindrical hole-forming tool, and allowing the cell mixture to solidify to produce a dermal construct;
3) removing the cylindrical hole-forming tool from the structure formed in step 2) to form cylindrical holes;
4) inoculating the fibroblasts or dermal papilla cells selected in step 1) into the cylindrical holes to produce a dermal layer;
5) inoculating keratinocytes onto the upper layer of the dermal layer to culture the cells until a cell sheet is formed; and
6) exposing the cell sheet to air and culturing the cell sheet to form an epidermal layer.

2. The method of claim 1, wherein the gene similar to the dermal papilla cells treated with the hair growth promoting substance exhibits an expression similar to dermal papilla cells when the dermal papilla cells are treated with the hair growth promoting substance as a result of heatmap analysis.

3. The method of claim 1, wherein the hair growth promoting substance is one or more selected from the group consisting of minoxidil, finasteride, CHIR99021, and CHIR98014.

4. The method of claim 1, wherein the gene similar to the dermal papilla cells treated with the hair growth promoting substance is one or more selected from the group consisting of AFF1, KCTD12, KANK1, NFAT5, RAPGEF6, ITGA2, TBX3, and ITGA6.

5. The method of claim 1, wherein the gene similar to the dermal papilla cells treated with the hair growth promoting substance is one or more selected from the group consisting of ITGA2, TBX3, and ITGA6.

6. A three-dimensionally cultured skin model produced according to claim 1.

7. A three-dimensionally cultured skin model comprising a dermal layer prepared by a composition comprising collagen and fibroblasts that exhibit gene expression similar to that of dermal papilla cells treated with a hair growth-promoting substance, and an epidermal layer formed by keratinocytes.
